(19) United States
(12) Patent Application Publication
(10) Pub. No.: US 2004/0096363 A1
(43) Pub. Date: May 20, 2004

(54) Point-of-care assay reader and analyzer

(76) Inventor: Larry Porter, Somerset, NJ (US)

Correspondence Address: MCHALE & SLAVIN, 4440 PGA BLVD, SUITE 402, PALM BEACH GARDENS, FL, 33410, US

(21) Appl. No.: 10/299,604

(22) Filed: Nov. 18, 2002

(51) Int. Cl. G01N 33/00

(52) U.S. Cl. 422/68.1

(57) ABSTRACT

The instant invention is point of care device for performing an assay test. The device is a reader employing a cartridge receptive to plain single window, plain double window, stick single window, and stick double window assay devices.

### FIELD OF THE INVENTION

The present invention relates to a point of care device that aides in providing a medical diagnosis or risk assessment for a patient using diagnostic tests or assays, and processing the information to give an indication of a medical condition or risk.

### BACKGROUND OF THE INVENTION

An immunoassay can be defined as any method using a preferential binding of an antigen with a second material, a binding partner, usually an antibody or another substance having an antigen binding site, which binds preferentially with an epitope of the antigen. The immunoassay methods include any known to those of skill in the art, including, but not limited to, sandwich, competition, agglutination or precipitation, for example.

Immunoassay technology provides simple and relatively quick means for determining the presence of analytes in a subject sample. The information provided from immunoassay tests are often critical to patient care. Assays are typically performed to detect the presence of particular analytes, such as antibodies that are present when a human subject has a particular disease or condition.

The advancement of immunoassay technology now allows for assay tests to be performed without the complex and expensive equipment used in hospitals and laboratory settings. Devices for performing assays are now available for point of care to quickly determine the presence of a disease or condition. Such devices typically provide qualitative results for the analyte or condition being tested for.

However, devices for qualitative analysis of assays are often prone to user error, and lack the accuracy of sophisticated equipment that perform and analyze the assays in hospitals and laboratories. For instance, assay devices often require the user to visually interpret an ongoing chemical reaction. In some applications, if the user mis-times reading the assay device by even a few minutes, the result of the assay may turn from negative to positive. Still, other devices fail to sufficiently distinguish positive from negative results.

Readers are provided in the known art for determining or analyzing the results of assays more accurately. In general, readers provide an improvement in that they may analyze an assay result, thereby removing subjective factors that cause human error. However, whereas readers may reduce operator subjectivity in reading or interpreting assay results, they do not help to control for or help mitigate other sources of assay variability. Such sources may include variability introduced by incorrect assay run times, uncontrolled reaction temperatures, or other possible operator-induced variability. The present invention addresses these and other shortcomings of the known art.

### SUMMARY OF THE INVENTION

The instant invention is a system for medical diagnosis or risk assessment for a patient at the point-of-care, such as in OTC field, occupational health centers, laboratories, emergency rooms, operating rooms, and doctor's offices. The system processes patient data, particularly data from point of care diagnostic tests or assays selected from the group of, plain single window, plain double window, stick single window, and stick double window test kits to provide an indication of a medical condition or risk or absence thereof.

The system includes a base unit for receipt of a test strip which includes an instrument for reading or evaluating the test data and software for converting the data into diagnostic or risk assessment information. The patient information includes data from biochemical tests, such as immunoassays, and from other procedures for the detection of medical conditions and illnesses that include infectious disease, fertility, abused drugs, cancer diagnostics and cardiac markers.

### BRIEF DESCRIPTION OF THE DRAWINGS

FIG. 1 is a top view of the point-of-care device;

FIG. 2 is a front perspective view of the point-of-care device with the cartridge in an open position; and

FIG. 3 is a perspective view of the point-of-care device with a test kit placed with the cartridge.

### DETAILED DESCRIPTION

The reader of the instant invention is a compact, portable device housed in a durable shell having dimensions about 20×20×10 cm with a weight less than 1.5 kg. The reader operates as a computing/data module having an internal RAM of about 20 Kbyte with 256 KByte Flashrom, External—32 KB RAM/32 KB EEPROM/512 MB Flash. An extended version has Internal: 20 K RAM/256 K Flashrom and External—1×128 K RAM/2×128 K EEPROM/512 K Flash. The use is powered by 4 alkaline batteries, type AA (approx. 2000 mAh) provides portability and will provide about one year use, assuming 100 measurements including 100 printouts, or at least 10 days of operation in the case of 10 series measurements per day. A LOW BAT message is being printed when the voltage level of the batteries falls below 4.5V. System shutoff will occur when the voltage drop below 3.6V. About ten measurements can take place between the LOW BAT message and system shutdown.

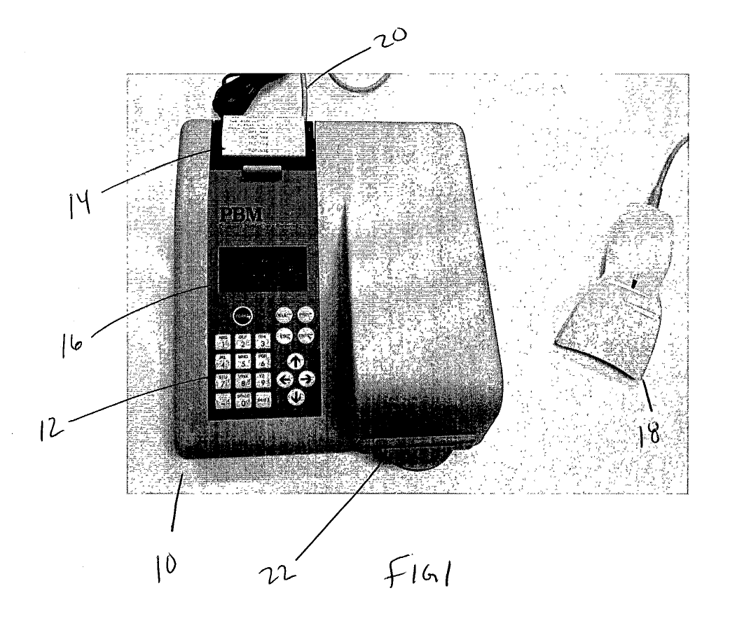

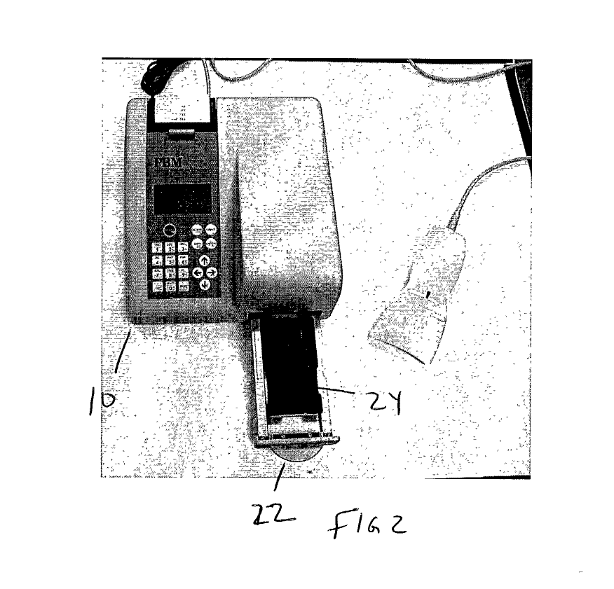

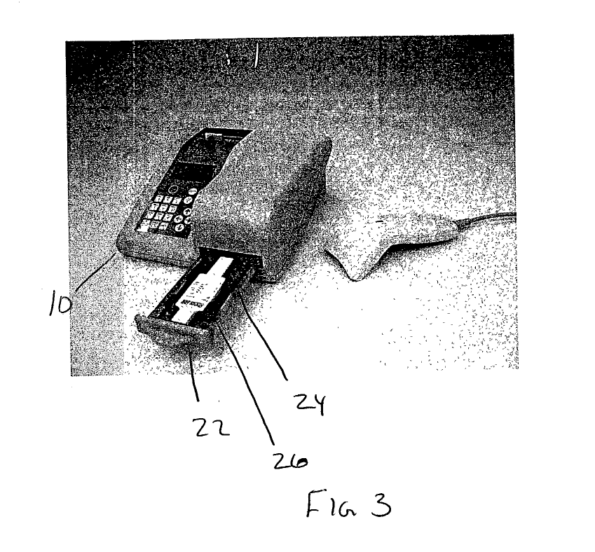

The device includes a means for accepting external power, namely, a connector for an external power supply employing an AC/DC converter 6V/2100 mA. An input of 90/120/264 V±10%, 50/60 Hz is converted to an output of 6 V±5%, 2100 mA, Ripple voltage <100 mV at 2100 mA with 90V<Ue<120V. Output: 6 V±5%, 2100 mA, Ripple voltage <75 mV at 2100 mA with 120V<Ue<260V. FIGS. 1-3 illustrate the point-of-care device 10 depicted a keypad 12, printer output 14, and display screen 16. An optical scanner 18 is coupled to the device 20 for use in the manual scanning of bar codes. Cartridge door 22 cover is manually opened for placement of a test kit FIG. 2 illustrates the device 10 with the cartridge door 22 opened wherein the drawer 24 is receptive to a test kit. FIG. 3 illustrates a test kit with a test kit 24.

Modification of the software system can be performed by downloading through the internet. The optical system includes two LEDs, preferably 591 nm yellow and a silicone photodiode detector. The preferred printer draws low amperage so as to extend battery life. Thermal printers, such as a Panasonic EPL-20502 are suitable.

Data to be displayed on LCD and printed includes the following: Patient ID, date and time, User ID, result. For instance, the LCD display and printout would include the following information:

### EXAMPLE NO. 1

| | |
|---|---|
| Name: | John Smith or (ID #: 1287DF) |
| Date: | Oct. 4, 2001 11:20 AM |
| Operator: | John Doe |
| Test: | DOA4 BM or (Lot # BM1G21) |
| Result: | Cont: Valid |
| | THC: Neg** |
| | OPI: Pos |
| | COC: Neg |
| | MET: Neg |

**Below the cutoff concentration, the signal will be above 13 peak height by CAMAG reading. The reader will express this as a negative. Above the cutoff, the signal will be equal or below 13, and the reader should express as a positive.

### EXAMPLE 2

| Name: | John Smith or (ID #: 1287DF) | |
|---|---|---|
| Date: | Oct. 4, 2001 11:20 AM | |
| Operator: | John Doe | |
| Test: | DOA10 CT or (Lot # CT1G21) | |
| Result: | Cont: Valid | Cont: Valid |
| | MET: Neg | BZO: Neg |
| | OPI: Neg | BAR: Neg |
| | COC: Pos | MTD: Neg |
| | THC: Neg | TCA: Neg |
| | PCP: Neg | AMP: Neg |

### EXAMPLE 3

| Name: | John Smith | |
|---|---|---|
| Date: | Oct. 4, 2001 11:20 AM | |
| Operator: | John Doe | |
| Test: | Cardiac EB or (Lot #: EB1H25) | |
| Result: | Cont: | Valid |
| | Myo: | 70.0 ng/ml |
| | CK-MB: | 15.0 ng/ml |
| | TnI: | 1.0 ng/ml |

**expression with 1/10 NG/ml

Alternative (semi quantitative)

| Name: | John Smith | |
|---|---|---|
| Date: | Oct. 4, 2001 11:20 AM | |
| Operator: | John Doe | |
| Test: | Cardiac EB or (Lot #: EB1H25) | |
| Result: | Cont: | Valid |
| | Myo: | Low (59.0-60.0 ng/ml**) |
| | CK-MB: | Neg (5.0-7.0 ng/ml) |
| | TnI: | Neg (0.1-0.4 ng/ml) |

**Use a different algorithm (software program) to detect the signal and expressed in the ranges.

### EXAMPLE 4

Test invalid

No control line detected

When control line intensity is below a certain level

Communication is by RS-232 or USB port. Data can be transferred automatically to a main hospital computer terminal when the unit is connected and linked to the main terminal and be accessible from the satellite lab and administration offices. LRE provides a protocol which can be linked to the host system.

Product recognition is performed by the reader: The product can be recognized by use of an infrared bar code detector (pen or gun style) tethered to the reader for use in scanning a bar code on the product box, detection to include the product lot number and expiration date. Alternatively the reader can read a bar code located on the assay device when inserted into the reader.

A numeric keypad is available for use in inputting patient ID and operator or product code). The reader will accept the following assay devices

Plain single window (stroke marker, cardiac, DOA)

Plain double window (DOA)

Stick single window (DOA)

Stick double window (DOA)

The operation of the reader includes the use of a Power on/off button, this key is inactivated while a measurement is being taken. An automatic shutdown mode is activated if 30 minutes elapses from the last operation or display of results. As a safety measure, the instrument also shuts down with a warning signal if the voltage drops below a safe operating level. The reader self-test checks the system to be sure it is operating within normal parameters. The test includes laser operation, internal standard range, battery power and checksum capabilities, all sets of data are checked with the checksum+meter and boot program.

Meter self testing occurs when the instrument is powered on and during the each sample test (only internal standard measurement). A Test procedure consists of the following steps:

1. Open the pouch and remove the assay device

2. Label the assay device with patient ID

3. Enter the product code by scanning the bar code on the kit box for product identification and lot # using a tethered scanner OR the product code, lot number and expiration date can be manually entered. Still another option the use of an instrument internal scan bar code on the device for product identification wherein:

1. The patient ID is entered using a keypad

2. Confirmation of the correct entry takes place requiring the user to press the enter key if correct.

3. Apply a sample and start timing (add a timer with multiple settings, possibly 10, for each sample on display?)

4. Press eject for cartridge (if cartridge is not in out position)

5. Place the assay device on the cartridge when the assay time is up

6. Press start (the reader pulls in the cartridge with the assay device on it)

7. Reader scans the result and displays the result on LCD Double devices have to be turned after reading the first part of the assay device.

8. The reader automatically prints out the result when reading is completed and sends data to the main terminal. The standard reading and printing time without incubation or delay time is 30 s.

As an option, if the assay time is over due, (e.g. prevent reading at 20 minutes instead of 15-minute point for the cardiac assay). Incubation time can be programmed individually to the test.

BioSite has coded chips, Reagent test, QC samples, program and Supervisor access.

Clock/Timer setting

Operator ID requirement

Password required to do QC functions (can also be transferred via PC)

Sorting and/or filtering or results and display—supports sorting in ascending or descending order of the selected result by filter type. Sorting by Sample ID, Test name, Date/Time. Results and operator ID.

Password protection—a password may be optionally set up by the system manager to restrict access to instrument use and deleting of results and data. (PassNumber!)

### Software Programs

| Product Name | Product Code |
|---|---|
| Plain single window | |
| Cardiac | |
| 3 in 1 (Myo/CK-MB/TnI) | EB |
| CK-MB/Myo | EW |
| TnI | ET |
| Stroke marker, | DOA |
| AccuSign 2 (COC/THC) | BT |
| AccuSign 3 (T/O/C) | BC |
| AccuSign 4 (T/O/C/A) | BA |
| AccuSign 4 (T/O/C/M) | BM |
| AccuSign 5 (A/O/C/T/P) | BE |
| AccuSign 5 (M/O/C/T/P) | BF |
| AccuSign 5 (T/O/C/BZ/A) | BG |
| Status DS 3 (T/O/C) | CC |
| Status DS 4 (T/O/C/A) | CA |
| Status DS 4 (T/O/C/M) | CM |
| Status DS 4 (T/O/BZ/A) | CZ |
| Status DS 5 (M/O/C/T/P) | CF |
| Plain double window | |
| Status 8 T/O/C/A/P/BZ/BA/M | CK |
| Status 8 T/O/C/A/P/BZ/BA/TCA | CD |
| Status 9 T/O/C/A/P/BZ/BA/TCA/M | CN |
| Status 10 T/O/C/A/P/BZ/BA/TCA/M/MTD | CT |
| Stick single window | |
| Status Stik 2 (THC/COC) Stick | BU |
| Status Stik 3 (T/O/C) Stick | CJ |
| Status Stik 4 (T/O/C/A) Stick | BV |
| Status Stik 4 (T/O/C/M) Stick | BS |
| Status Stik 5 (A/O/C/T/P) Stick | BW |
| Status Stik 5 (M/O/C/T/P) Stick | BX |

Stick double window

Approximately 500 test results can be stored in the memory. The results are stored in sequential records until all measure records are filled. At that time, a warning will be displayed and the next data recorded will overwrite the oldest data stored. Asking the operator to confirm the memory clear operation will always precede the test result memory clear function. All stored data can be recalled through user guided prompts. Results can be displayed or printed.

Alarms

Both audio and visual indications are provided for

Power loss

Communication error

Measurement error

Operational error

Computer system error

Low printing paper

Memory is full

Slope out of range

Calibration: The instrument has an internal standard to eliminate electronic and optical drift and offsets. This is used for performance checking of the instrument as well as correction. To determine if the instrument is still within tolerance, the standard is measured on a regular basis to check that it is within preset factory calibration limits. The instrument will perform a self-test function on power-up to verify all subsystems are functioning properly.

### Software Flow chart

On-Off Button

When first turned on the reader will go through a diagnostic check of optics and functions, LCD display will indicate diagnostic completed successfully or an error message will appear. One message may be “memory is full”. If you wish to store results, please do now. Reader will delete the oldest results first if memory full. Another message might be battery is low.

1. Screen 1

A. Run Test

A. Run QC

B. Retrieve QC Results Goes to C.1.d

C. Retrieve Patient Results Goes to C.4

E. Supervisor

D. Supervisor Authorization and Specifications set-up Password ____

1. QC Parameters

a. When will QC be performed, complete one

Each new lot of kits __

Each Day __

Every ___ tests

Every ___ days

Operator Option __

b. Controls to be Run indicate each to be run

Neg. _

Low Pos. _

Mid Pos. _

High Pos. _

c. Control expected results

TnI

Control Limits

Neg. 0−1.4

Pos. >1.4

CK-MB

Control Limits

Neg. 0-4.9

Pos. >4.9

Myoglobin

Control Limits

Neg. 0-49.0

Pos. >49.0

d. Retrieve QC results Yes No Supervisor must pre-authorize Operator

By Test Group ____ Cardiac, drugs of abuse, etc.

By Lot ____

By Date from _/_/_ to _/_/_

By Control

Lot #____

Date _/_/_

Down Load/Save Results to Computer

QC Results

All

By Date from _/_/_ to _/_/_

By Lot # from ____ to ____

Delete these results from Reader Yes No

Are You Sure? Yes No

5. Assign Operator 4 digit Passwords

Operator 1 ___

Operator 2 ___

Operator 4 ___

6. QC results if out of specification

a. Supervisor must Over-ride __

b. Operator Over-ride __

Over-ride “Yes NO”

Confirm Over-ride

Comment required ______

7. Retrieve Patient Results Yes No Supervisor must pre-authorize Operator

BY ID #___

By Name ______

By Date from _/_/_ to _/_/_

Dow Load/Save to Computer

a. Patients

All

By Date _/_/_ to _/_/_

A. Run Test

1. Enter Password ___

2. Enter Test to be Run ___ 2 digit letters or select from menu

3. Enter or wand Test Lot #____

8. Enter or wand Expiration Date ____ If product is expired test can not be run. If lot not entered in C.1.c, Supervisor must enter before test can be run.

9. Enter or wand calibration curve #___ (Optional)

10. Run QC will appear if: set parameter C.1.a has been realized before Test can be run.

A. if run QC

B. Enter control lot numbers and expiration date or Barcode wand in:

Neg. Control lot #____ Expiry _/_/_

Pos. Control Lot #____ Expiry _/_/_

7. QC Out—Menu goes to C.3.a or C.3.b

8. Enter or wand patient ID #______ (Optional)

9. Enter or wand patient name ______ (Optional)

10. Incubation

a. Onboard Reader _

b. Incubate off Reader _

11. Read

12. Print Results

13. Down Load/Save Results to Computer

a. Patients

All

By Date _/_/_ to _/_/_

a. QC Results

All

By Date from _/_/_ to _/_/_

By Lot # from ____ to ____

Delete these results from Reader Yes No

Are You Sure? Yes No

Print Out Example

Patient ID #______ If Control Results: Lot #______

Patient Name ______ Expiry Date _/_/_

Date Run _/_/_

Operator (from password) ___

Test Run ______

Test Lot #______

Test Expiry _/_/_

Test Results

THC Neg

OPI Neg

COC Pos. (Bold Print)

AMP Neg

Note: If no control line is present, it states Test Results “Invalid” (bold)

Operator Signature/Date ______/___ Turn Off

What is claimed is:

1. A point of care device for determining results from a diagnostic assay device, said point of care device comprising: a base housing; a cartridge slidably insertable into said housing, said cartridge size to receive assay devices selected from the group of, plain single window, plain double window, stick single window, and stick double window; at least one LED and photodiode detector operatively associated with said cartridge and arranged to read data from said assay device; a control unit having a processor for analyzing said data; and a screen for displaying said data.
